(19) United States
(12) Patent Application Publication
(10) Pub. No.: US 2015/0104496 A1
(43) Pub. Date: Apr. 16, 2015

(54) COMPOSITION CONTAINING AT LEAST ONE NUTRIVITE, AT LEAST ONE DISINFECTING OR DECONTAMINATING, AND/OR AT LEAST ONE PROTEASE-INHIBITING ACTIVE COMPOUND AND/OR ACTIVE COMPOUND COMPLEX

(71) Applicant: Birgit RIESINGER, Munster (DE)

(72) Inventor: Birgit RIESINGER, Munster (DE)

(21) Appl. No.: 14/519,495

(22) Filed: Oct. 21, 2014

Related U.S. Application Data
(63) Continuation of application No. 12/667,356, filed on Jun. 16, 2010, now abandoned.

(30) Foreign Application Priority Data
Jul. 3, 2007 (DE) 10 2007 030 931.9

(51) Int. Cl. A61K 31/444; A61L 15/60

(52) U.S. Cl. CPC A61K 31/444; A61L 15/60; A61L 2300/204; A61L 2300/404
USPC 424/445; 514/332

(57) ABSTRACT

The present invention relates to a composition containing at least one nutritive, at least one disinfecting or decontaminating and/or at least one protease-inhibiting active compound and/or active compound complex for the external care and/or treatment of wounds of the human or animal body.

### CROSS-REFERENCE TO RELATED APPLICATIONS

The present application claims priority from and is a continuation from U.S. patent application Ser. No. 12/667,356, filed Jun. 16, 2010; which is a U.S. National Stage from PCT Application No. PCT/EP2008/058421, filed Jul. 1, 2008, which claims priority from German Application No. 10 2007 030 931.9, filed Jul. 3, 2007, all herein incorporated by reference in their entireties.

### BACKGROUND

The present invention is comprised of a composition for external care and/or treatment of wounds in accordance with the concept of claim 1.

Many animal and human wounds, particularly chronic, i.e. wounds lasting several weeks, or wounds with fistulas, typically heal more slowly. Most require a replacement of lost tissue with renewable tissue from the affected area. A characteristic of such wounds is the development of pathological exudates, which damage and delay the healing process. Such wounds are particularly prevalent in the extremities (particularly the venous ulcer), in the back (decubitus), but also in the oral region (dental and jaw surgery wounds).

Regarding the inhibitory effects, which result from the pathological exudate in the wound, thereby extending the chronification process, the removal of the pathological exudate is necessary for the wound to heal. The state of technology describes dressings containing super-absorbent particles or PU foams which have a high absorbency capacity. These remove the exudate from the wound, and restrain such. Due to the high level of suction, the exudate is not only absorbed from the surface of the wound, but also from the depths of the wound. In this manner, the affected area and surrounding region is better protected from active damage due to proteases. Such dressings are familiar to the applicant of the present invention from, for example, the WO03094813.

Due to their high absorbency capacity, these dressings may remain on the wound for longer, i.e. several days, periods of time. The resting period of the wound is significantly extended, and bothersome and painful dressing changes are thereby reduced. This contributes to an improvement of the wound situation, in that through the reduced dressing changing frequency, the cooling of the wound as well as the risk of infection and the danger of secondary trauma are decreased.

The long resting period may however lead to septic processes, as the wound is cleaned and/or disinfected less frequently. Particularly in certain cases, one must expect microorganisms in the dressing to reproduce, and thereby result in a secondary re-infection of the wound.

In addition, particularly chronic wounds suffer from nutrient deficits, which significantly slow down and influence the healing process.

These problems specified particularly for chronic wounds have as yet not been addressed by the dressings.

### SUMMARY OF THE INVENTION

Provided herein are systems, methods and compositions containing at least one nutritive, at least one disinfecting or decontaminating and/or at least one protease-inhibiting active compound and/or active compound complex for the external care and/or treatment of wounds of the human or animal body.

The systems, methods and compositions are set forth in part in the description which follows, and in part will be obvious from the description, or can be learned by practice of the methods, apparatuses, and systems. The advantages of the systems, methods and compositions will be realized and attained by means of the elements and combinations particularly pointed out in the appended claims. It is to be understood that both the foregoing general description and the following detailed description are exemplary and explanatory only and are not restrictive of the systems, methods and compositions, as claimed.

### BRIEF DESCRIPTION OF THE DRAWINGS

In the accompanying figures, like elements are identified by like reference numerals among the several preferred embodiments of the present invention.

FIG. 1a is a graph showing the absorption of exudate in a, for the most part, dry, absorbent dressing containing super-absorbent polymers.

FIG. 1b is a graph showing the exudate absorption of a dressing containing super-absorbent polymers, moistened with Ringer's solution.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

The task of the present invention is therefore, to provide a composition, which makes it possible to confront the septic processes in chronic wounds having moderate to strong exudation.

A further task is to provide a composition which enables the promotion of the healing process for chronic wounds having moderate to strong exudation.

A further task is to provide a dressing which may be applied for a longer period of time to a wound, without the risk of said application resulting in a septic process.

A further task is to provide a composition which reduces the activity of healing inhibiting proteases.

These tasks are solved through the characteristics of the present set of claims. The subsidiary claims provide preferred design versions. It should be noted thereby that the specified range specifications are to be understood consistently, including the respective limit values.

In accordance with this, a composition is intended which contains at least one nutritional and/or at least one disinfecting agent and/or complex of agents for external care and/or treatment of wounds to human or animal bodies.

The term “complex of agents” should not be understood in the following as a complex in the chemical sense, but rather, particularly, as a synergistic composition of effective agents

In the publications, the relationships between the nutritional condition of the patient and the healing have already been described (Arnold M., Barbul A. (200), “Nutrition and wound healing”, Plast. Reconstr. Surg. 117(7 Suppl.): pages 42-58). Similarly, it has been indicated that by improving the nutritional condition of a patient, the healing process may be improved (Patten J. A. (1995) “Nutrition and wound healing”, Compend. Contin. Educ. Dent. 16(2):200, 202-4, 206-8). However, the methods described in the publications as a means of treating this deficit, a systemic approach is always recommended, i.e. compensation for the deficit by improving the nutritional situation. Topical approaches in this regard have not yet been described in the available literature.

The reasons for this are that the experts avoid, in general, a so-called “off label use”, i.e. the use on a wound of familiar, or even agents which appear to be trivial, not having a familiar indication for use. In addition, there are major legal problems regarding this.

Furthermore, according to the prevalent school of thought, a wound should be treated with as few as possible, and with precisely defined, means. The experts, however, are opposed to the use of complexes and, if the case may be, less defined compositions being used in the treatment of wounds.

In addition there is the aspect, that agents for improving the nutritional condition of a patient that may be taken orally are not necessarily suitable for topical treatment, as they may cause allergic and/or immunity reactions, or respectively, infectious processes. As a result, only hypoallergenic, non-pyrogenic, and, as the case may be, sterile ingredients, may be used, which are naturally much more expensive to produce.

A wound treatment article for treating local pain in a wound is known of from the WO03055536, which contains a composition with a means for absorption of exudates from wounds as well as a pain reliever, whereby the composition is a means of inhibiting infections and relieving pain.

A product with these characteristics, having with the brand name “Biatain-Ibu” from the company Coloplast, is also available. With this application, or respectively, this product, the pain relief aspect is the primary focus, however, and not the promotion of the healing process, which is significantly more complex than pain relief.

Furthermore, in accordance with the invention, the use of one or more compositions of this sort is intended for the production of a means for external, non-systemic topical caring for and/or treating of wounds to human or animal bodies.

Particularly preferred thereby, it is intended that the dosage form of the, at least one, agent or complex of agents be selected such that it results in only a topical, non-systemic effect.

In the following, the term “non-systemic” should be understood to mean that the, at least one, agent has only a local and/or topical effect to the affected area.

This can be accomplished for example, through a suitable determination of the concentration and/or dosage of the, at least one, agent. This will be explained more precisely later in this text.

Furthermore, it is intended preferably, that the, at least one, agent or complex of agents be available in a form, such that when applied directly or indirectly to the wound said agent may migrate into the wound and/or cells in the surrounding region. It is intended that there be a gradual, step-by-step application of the healing agents over the course of the application with a reduction of the concentration and dosage of said agents in the dressing to the affected area.

Furthermore, it is intended in one design version that at least one agent or complex of agents be provided in a form, which allows for admission of said through the cells of the affected area.

The term “direct application” should be understood to mean that the composition is applied to the wound directly in the form, as the case may be, of a powder, a fluid, a paste, or a gel.

The term “indirect application” should be understood to mean however, that the composition is contained, for example, in a dressing or another medium, and said dressing or other medium is then applied to the wound.

In the following, the term “migration” should be understood to refer to passive and active transportation processes, which affect an application of the, at least one, agent or complex of agents in the wound. These are, particularly, diffusion processes, but also active transportation processes as well as mechanical effects to, as the case may be, a present dressing, which result in the movement of a fluid from the dressing into the wound.

Exudate is a wound fluid derived from plasma through the infection process of the wound edema. Just as the blood is responsible for the transportation of nutrition and other substances to be supplied to various parts of the body, in a similar manner, the exudate serves as a means of caring for the affected area, and the healing cycle. In order to fulfill these many functions, it contains a wide range of components, which, as a result has a specific mass which is slightly higher than that of water. In this manner, it differs from transudate, which is the result of non-infectious processes, and has a significantly lower specific mass, with a lower cell and protein content. Aside from providing nutrition to fibroblasts and epithelial cells, the exudate coordinates the various processes of the healing process both temporally and spatially through its high content of growth factors and cytokines. These are comprised primarily of platelets, keratinocytes, macrophages and fibroblasts. They influence the motility, migration and proliferation of the various cells involved in the healing process. In this manner, the immigration of cells to the affected area is promoted just as much as the care for the newly developed granular tissue as a result of angiogenesis. The cleansing of the wound is also supported by the exudate. It contains various serine, cysteine, and aspartic proteases as well as matrix metalloproteinases, which are active in the removal of irreversibly damaged tissue and thereby prepare the affected area for the subsequent phases of the healing process.

Components of the physiological exudate are, particularly, salt, glucose, cytokines and growth factors, plasma proteins, proteases (particularly matrix metalloproteinases), granulocytes and macrophages.

If there is not significant progress in the healing process within a few weeks, in regard to the various phases of the healing process, then one refers to this as a chronic wound. In this case, one observes longer exudative phases as complications, and refers to this as pathological exudation, which may contribute to a chronification of the wound. The underlying causes are for the most part complex and may also be of a systemic nature. It is, however, not surprising that based on the previously explained importance of the exudate for the healing process, that complications in the healing process are reflected by a significantly altered composition and effect of the exudate. Among others, through a shift in the concentration of the individual components of the exudate, the exudate loses its positive effect of normally promoting healing in the case of chronic wounds. In particular, the content of inflammatory cytokines and proteases is significantly increased in pathological exudates. The content of growth factors, however, is decreased.

A particularly serious difference occurs in regard to the activity of the previously referred to proteases. Aside from the preparation of the affected area, they are also involved in the later conversion of granular tissue to scar tissue. These enzymes are normally formed as an inactive pre-enzyme, and its activation is regulated by respective inhibitors (“tissue inhibitors of metalloproteinases, TIMPs”), which at the same time have a positive effect on cell growth. In chronic exudate it would seem that, due to disturbances in the regulatory system, the activity of the proteases is increased, which contributes to active regression of the wound.

The terminology “nutritional substance, or respectively, complex of substances” should be understood in the following to mean macro and micro nutrients, particularly minerals, trace elements, vitamins, pro-vitamins, vitamin derivatives, proteins, proteinogenic and non-proteinogenic amino acids, carbohydrates, particularly starch, glucose and fructose, fats, fatty acids and fatty substances (particularly mono- and diglyceride, phospholipids, lipids, cholesterol, squalene, carotene), organic acids, nucleic acids and/or primary or secondary vegetable material.

The term “disinfecting substance or complex of substances” should be understood in the following to mean antiseptic, antibiotic, bacteriostatic, antiviral, and/or antimycotic substances. For this, it may be seen that the antiseptic, antibiotic, bacteriostatic, antiviral and/or antimycotic effect only refers to certain representatives of the respective class of organism (bacteria, virus and/or fungus), particularly pathogenic representatives, while other representatives, particularly non-pathogenic representatives, are not meant.

The term “decontaminating substance or complex of substances” should be understood to mean, in the following, substances that serve as decontaminants. Decontamination has the purpose of removing potentially damaging substances from the surface area. The decontamination is successful when the particles are removed from the surface area and can be appropriately disposed of. For the decontamination of a chronic wound, there exists only a very limited range of possibilities. Requirements for the decontamination of a chronic wound consist of, for one, the removal of cell detritus, dried secretions, and if the case may be, dead tissue (necroses). Furthermore, in the framework of decontamination, micro-organisms, as a rule bacteria, sitting on and within the tissues are removed. The aim of the decontamination is to keep the bacterial count and the contamination of the wound so low that the natural healing process is not inhibited.

The background for this approach—in contrary to what was said at the beginning—is not necessarily chronic wounds, but also acute traumata, such as may be observed, for example, in military operations, resulting, for example, from irradiation, burning or the influence of biological weapons. Particularly with biological weapons, the decontamination characteristics are of primary importance. In addition, there is the fact that the super-absorbent polymers, which will be described later, can absorb and immobilize micro-organisms, particularly those from biological weapons. A dressing containing disinfecting substances as well as super-absorbent polymers can therefore be used in the first aid of a biologically contaminated individual, in particular, a soldier. It must be properly disposed of after use—i.e. autoclaved, as a rule.

With burning and irradiation, which also are often observed in connection with military conflicts, tissue necrosis occurs, which may be superficial to some extent, and to some extent however is embedded deep in the tissue, and can make its way to the surface through fistulas. Wounds of this sort are extremely susceptible to infection. For this, a dressing containing a disinfecting composition as well as super-absorbent polymers can also be extremely useful. In addition, in that such a dressing is capable of this, necrotic components as well as, if the case may be, irradiated particles—particularly those making their way to the surface through fistulas—may be removed from the affected area, and restrained. A dressing of this sort must also be properly disposed of.

The decontaminating effect can be affected or increased through the application of necrolytic and/or exudation forming components, as will be described later. The promotion of the necrolysis facilitates thereby the deconstruction and removal of contaminated components, while the promotion of the exudation facilitates the cleansing and thereby the decontamination.

The term “proteases inhibiting substance, or complex of substances” should be understood in the following to refer to such substances that have an inhibiting effect on proteases in the affected area, particularly matrix metalloproteinases. These may be, particularly, acidifying substances or complexes of substances, as well as protease inhibitors, super-absorbent polymers, chelators for divalent cations, collagen, or coated magnetic particles.

The term “acidifying substance or complex of substances” should be understood in the following to mean such substances as those having an acidifying effect to the wound, i.e. lowering the pH value in the wound. This can be useful in inhibiting the damaging proteases in certain phases, as is described above, which are normally active in the neutral to alkaline region. “Acidifying substances or complexes of substances” may therefore refer to, for example, acids, and particularly organic acids. Furthermore, these can be non-pathogenic, acid producing bacteria, particularly lactic acid bacteria, pre-biotics, i.e. such acid producing bacteria that are a part of a selective nutritional substrate which can be metabolized, or are symbiotic (see below).

The substances or complexes of substances are preferably water soluble, as previously mentioned. This means in particular as well, that they are preferably soluble in exudate.

A stronger wound exudation results in a higher dissolving rate, based on the above mentioned solubility, and thereby, a larger quantity of substance delivered to the wound. This is particularly beneficial if there are several wounds, whereby the necessity for treatment increases in proportion to the degree of pathological exudate. In this case, a self-regulating mechanism is initiated, as a result of the design of the invention, whereby the wound “self extracts” the amount of substance needed according to its degree of exudation, in the sense of the above definition.

It can thereby be intended that a light exudation first results in the composition assuming the function of absorbing exudate, which may be beneficial for the time being. If, over time, the exudation increases, the components of the composition are released, and are transported to the affected area of the wound by the reflux (sustained-release effect).

In another design, it may be intended that the substances or complexes of substances at least partially are in a fat-soluble form. This may be beneficial if fatty or non-polar mediums are used, such as, for example, Vaseline, the petroleum based blistering compound, or a fat-gauze.

Furthermore, in accordance with the invention, it is intended that the, at least one, substance or complex of substances be introduced to a water soluble matrix. Such a matrix can, for example, have the form of water soluble sheets. If this matrix is applied to the wound, it dissolves without a trace, and the substances or complexes of substances are able to take effect. Suitable materials for such a sheet, or, respectively, such a matrix are, for example, polysaccharides, such as modified (i.e. water soluble) starches, using an unloading structure, or gelatin.

Particularly preferably, it is intended that the, at least one, substance or complex of substances, be incorporated in a planar pad.

This pad may be, for example, an alginate mat, a pad or layer of carboxymethyl cellulose, air-laid, fleece, PU, silk or cellulose. In particular, it may also be a wet wipe, which is impregnated with a solution containing the composition of the invention. In this design, an additional dressing is normally necessary, which is used to keep the pad in place on the wound. A dressing of this sort is described, for example, in the DE102007019622 by the applicant, the disclosure content of which is to be added in full to the disclosure content of the present invention.

This dressing can be, for example, a dressing for absorbing exudates from wounds, as the applicant's DE10059439 as well as WO03094813 describes for the present invention, the contents of which are to be added in full to the disclosure content of the present invention.

The size of the pad may correspond basically to the area of the wound, in order to provide the wound with the said substances or complexes of substances as homogenously as possible. Alginate (or alginic acid, E400) is formed in the cell walls by brown algae, and places the structuring element in the alga. Alginate is used primarily as a thickening or gelling agent. The gelling is a result of storing calcium ions, from which three-dimensional structures are formed. Although this reaction with calcium takes place quite quickly, in practice various methods are used in order to control the rate of the reaction. For this, poorly soluble calcium salts are used, which gradually release the calcium through slow acidification. Furthermore, sequestrants may also be used, which can bond to a portion of the calcium.

The specified substances or complexes of substances may preferably be applied to a pad of alginate. The light, minimal swelling of alginate allows for a particularly good adaptation to the affected area with a particularly high application and contact surface on the surface of the wound, such that in this case a particularly intensive exchange of nutrients is able to take place in the affected area. The alginate pad, or respectively, another material compatible with the wound may have a meandering, labyrinthine, spiral or radial incision, which allows for an easier application to the affected area.

Carboxymethyl cellulose (CMC) is a cellulose derivative, whereby a portion of the hydroxyl groups of the cellulose is combined as ether with a CH2-COOH-(carboxymethyl-) group. For the production, the cellulose is transferred to reactive alkaline cellulose and subsequently converted to carboxymethyl cellulose with chloroacetic acid. The cellulose structure remains intact. CMC is insoluble in water in its acidic form. It is however relatively water soluble in alkaline states.

Mats of carboxymethyl cellulose provide for a good adaptation to the affected area and show a high affinity to water; therefore, the potential for introducing other substances into the CMC pad through water is to be emphasized. In this manner, substances or complexes of substances which are to be released to the surface of the wound, particularly in the case of over-saturation with exudate, can be washed out of the pad, and thereby made available.

PU pads are foam dressings of polyurethane, having the characteristic of being able to absorb water using the principle of capillary forces, and thereby expand. When these contain the said substances or complexes of substances, the low retention force of the foam when moistened with exudate, for example, leads to a rinsing of the substances or complexes of substances in the wound.

Fleece is a textile material, which in contrast to textiles made of yarns, consists of single, non-woven fibers. Fleece differs from paper generally in the length of the fibers, which are much shorter in said. Fleeces generally absorb liquids quite well.

Frequently, the specified materials are produced as air-laids. This process results in very smooth and absorbent products, frequently having a three-dimensional surface which is ideal for use with the composition of the invention.

The composition of the invention can ideally be presented in the form whereby it is applied to a medium of silk and/or hydro-fibers and/or incorporated in an impregnated bandage, embedded in colloid, embedded in silicone, embedded in CMC, embedded in polyester film and/or embedded in polyethylene film.

Silk is a fine textile fiber extracted from the cocoon of the silk worm, the larvae of the silk spider. Due to its hygroscopic characteristics in non-woven layers, the release of the substances into the affected area is improved.

Hydro-fibers are produced from sodium CMC and polyester fibers and can contain large quantities of liquids. The liquid is absorbed into the fibers and spreads very little horizontally, and thus the danger of maceration is quite minimal. A moist, warm healing promoting environment is formed beneath the dressing. It is ideal, in combination with the composition of the invention, for exudate management and the promotion of granulation.

Impregnated gauze is gauze that is impregnated with a substance such as Vaseline, wax or oil in order to inhibit the sticking of the gauze to the affected area.

Moreover, the coating is suited to storing fat-soluble substances and/or complexes of substances in accordance with the invention. These are successively released upon contact with the affected area, and transferred to the wound.

A colloid is a system comprised of clusters or small solid bodies that are finely distributed within a medium. The particles of this so-called colloid dispersion phase are generally 1-1,000 nm in at least one dimension. The medium itself is referred to as a dispersion medium. The field of chemistry which is concerned with colloids is called colloid chemistry. Dispersions comprise one category of one fluid in another fluid, in that the two cannot mix. Examples of these so-called emulsions are to be found in cosmetics. When dealing with more than two substances, so-called multiple colloids, one refers to multiple emulsions.

Colloids have an enormous internal surface area, which is highly beneficial, particularly with substances that are activated on contact (as is the case, for example, with anti-bacterial functioning colloidal silver ions). The technologies for producing such colloids are in the field of nanotechnology.

Silicone is a term for a group of synthetic polymers, whereby silicon atoms are coupled by oxygen into molecule chains and/or networks. The excess free valence electrons of the silicon are saturated by hydrocarbon radicals (for the most part methyl groups). As a rule, silicones are physiologically compatible (not harmful), and for this reason are used for skin protection, cosmetic skin care and plastic surgery.

Polyesters (PE) are polymers with ester bonds —[CO—O—]— in their main chains. They may be used within the framework of the present invention as inert mediums without retention force to the substances and/or complexes of substances.

Polyethylene (PET) is a thermoplastic synthetic produced by the polymerization of ethane [CH2=CH2]. Films of PET may be used within the framework of the present invention as inert mediums without retention force to the substances and/or complexes of substances. For this purpose, other thermoplastic synthetics, such as polyester or polyamide, may also be suited.

In another preferred design, it is intended that the, at least one, substance or complex of substances, is integrated in a protective dressing.

A protective dressing is in direct contact to a wound, and prevents a secondary dressing (e.g. a dressing containing super-absorbers) applied to the protective dressing, from sticking to the wound. A protective dressing of this sort—not, however, having the composition of the invention—is known of, for example, under the brand name “Sorbion Plus.” The advantage of this design is that the protective dressing is in direct contact with the wound, and the substances of the composition can therefore be brought into direct contact with the affected area, thereby avoiding long diffusion paths. Further advantageous designs of a protective dressing of this type are for example, gauzes containing oils, (lipo-) colloids, silicone, or carboxymethyl cellulose. A protective dressing of this type is already known of from the DE102006017194 by the applicant, the disclosure content of which is to be added in full to the disclosure content of the present invention. This consists of a film made of thermoplastics, having several three-dimensional perforations, the sides of which are, for the first surface, smooth, in each case beginning with an overlapping border having a free edge, and the second surface of which is rough and easily grasped. This protective dressing has cavities on one side, in which the substances of the composition can be placed in advance, and thereby applied directly to the affected area.

Furthermore, it may be intended that the, at least one, substance or complex of substances be incorporated in an absorbing dressing having at least one active element, selected from the group containing: a foam pad, an air-laid, a carboxymethyl cellulose pad, an alginate pad and/or a pad containing super-absorbing particles.

The specified foam pad may be, for example, a pad of polyurethane foam.

Super-absorbing particles (also called super-absorbent polymers) are synthetics which are able to absorb many times their weight in fluid. The product comes in the form of a white, coarse powder having particle sizes ranging from 100-1,000 μm (=0.1-1.0 mm). It is found, for the most part, in diapers, but also in products for feminine hygiene and incontinence hygiene. As a rule, super-absorbents are chemically comprised of copolymers of acrylic acid (propenoic acid, C₃H₄O₂) and sodium acrylate (sodium salt of the acrylic acid, NaC₃H₃O₂), whereby the proportions of the two monomers may vary with regard to each other. In addition, a so-called core cross linker (CXL) of the monomer solution is added, which binds in places the long-chain polymer molecules which are produced to each other with chemical bridges (they are “netted”).

This dressing ideally contains 10-60% by mass of this super-absorber (SAP). It is preferably designed such that it contains no binder. For this, ideally it is intended that the core of the dressing is comprised of cut cellulose fibers, or, respectively, layers, which are in contact with the SAP particles at a slant, and in this manner stabilizes the dressing.

Ideally, this dressing has two cellulose pads on both sides of this core, as well as, as the case may be, a sheath made of material that allows fluid to pass through it.

A dressing which contains super-absorbent particles is described in, for example, the DE10059439 or in the EP1507498 by the applicant of the present invention, the contents of which are to be added in full to the disclosure contents of the present invention.

A dressing of this sort is an absorption body to be applied to the human body, in particular for absorbing fluids which are secreted from parts of the human body, such as wounds. These absorption bodies are comprised of a, for the most part, flat piece of material made of absorbent material, comprised of an absorbing fleece containing a dispersion of super-absorbing particles, and a sheath made of a material which allows fluid to flow through, which contains the piece of material and creates a barrier against solid excretions, and allows the passage of other secreted substances to the absorptent material located within the sheath. A dressing of this type is available, for example, from the company Sorbion, of Germany, under the brand name “sorbion sachet”.

It is particularly suited to hydroactive therapy of chronic wounds, in which harmful exudate, including the harmful components contained therein (particularly pathogens and proteases), are absorbed and contained by the super-absorbers, while simultaneously generating a healing promoting moist environment. In addition, dressings of this sort, due to their high absorption capacity, may remain on the wound for longer periods of time, thereby preventing traumata associated with frequent changes of the dressing. The specified dressings have been very successful in the treatment of chronic wounds exhibiting strong exudation.

The previously mentioned dressings may also be designed such that the actual absorbing material is available in the form of a powder, granulate, flakes, beads, or shreds. In this manner, the contact region between the exudate and the absorbing material is enlarged, and the absorption of exudate, as well as the release of the substances or complexes of substances of the composition of the invention to the wound is thereby accelerated. Furthermore, the dressing, of any pad or particle components, may be concave in shape.

Particularly preferred is a design whereby the, at least one, substance or complex of substances is incorporated in a dressing containing super-absorbent particles as well as at least one foam pad and/or carboxymethyl cellulose pad.

A dressing in various forms of this configuration is described, for example, in the application WO2007051599 by the applicant of the present invention, the contents of which is to be added in full to the contents of the disclosure of the present invention. The same applies to the DE20200601682 by the applicant of the present invention.

In addition, the combination of a dressing containing super-absorbent polymers and a composition in accordance with the invention with a vacuum drainage device is also intended.

Dressings of this sort—as yet without the composition of the invention—are familiar to the applicant of the present invention from, in particular, the patent applications WO200604240 and WO2006056294.

With dressings of this sort, whereby as a rule they are applied to the wound in a manner such that they are airtight, and attached to a vacuum pump, the drainage effect is of fundamental importance. However, the vacuum pump does not run continuously, but, rather, is turned off periodically. In these phases, the healing promoting effect of the composition of the invention could become significant.

In this manner, a sandwich construction, for example, of a cellulose pad containing super-absorbent particles with high absorption capacities, as well as a foam pad with relatively limited absorption capacities, can be created. A dressing of this sort allows for the accommodation of the absorption capacity or the degree exudation in accordance with the type of wound, through the selection of the appropriate side to be applied.

These measures allow for the prevention of the frequent phenomenon of wound maceration, which occur when a dressing with limited absorption capacities (such as with, for example, a pure foam pad) is over-saturated with exudate.

In particular, it can furthermore be intended that, in the case that it is so intended, it be designed such that active substances, at least partially, remain in the dressing. In this manner, a bacterial growth in the dressing as a result of absorbed exudate which contains bacteria may be prevented. This last process is of particular significance because some of the dressings specified—particularly those containing super-absorbers—absorb large quantities of fluid, and therefore may be left on the wound for long periods of time. Because the dressing is sterile, and in accordance with the invention contains nutrients, the potential for an enhanced bacterial growth would otherwise need to be accounted for.

In addition, the exudate takes on two roles in said design version. On one hand, it is in many cases a fluid for removal, which, by absorption in the dressing, significantly promotes the healing process. On the other hand, the exudate serves as a dissolving agent for the substances or complexes of substances mentioned, and contributes thereby to the dissolving of said, and the introduction, or diffusion, at least in part, to the wound. This last process is of particular significance because some of the dressings mentioned—particularly those dressings which contain super-absorbers—absorb large quantities of fluid, and can, therefore, remain on the wound for a long period of time. As a result, in many cases, enough time is available for diffusion processes.

As a result of delayed solubility of the substances or complexes of substances mentioned, a sustained-release effect, i.e. a treatment of the wound over a longer period of time, is enabled.

In a further, alternative design version, it is intended that the, at least one, substance or complex of substances be introduced in a solution.

Such a solution may be used, for example, for a defined moistening of the dressings, in particular for such dressings that contain super-absorbent particles. In this manner, on the one hand, a healing promoting, moist environment is generated, and on the other hand, the nutritional and/or disinfecting functions are observed. A sustained-release effect can also be observed with a dressing which has been prepared in this manner, i.e. a gradual treatment of the wound is obtained over a longer period of time.

In particular, water, but also, for example, a physiological saline solution, Ringer's solution, blood serum, or plasma, may be used as a dissolving agent.

There are dressings known of that contain super-absorbing particles and are impregnated with a physiological solution that are used as cleansing dressings for wounds. These solutions are so-called Ringer's solutions, having an electrolyte composition corresponding to the plasma of the subject (i.e. 8.60 g NaCl, 0.30 g KCl and 0.33 g CaCl₂). This is not a solution with nutritional or even disinfecting properties, as the substance quantities selected are quite small, and insufficient in their composition. In addition, the use of topical medicines or disinfecting agents in combination with such dressings is expressly advised against.

Additionally, these dressings are primarily for cleansing purposes, and do not correspond to treatment as defined in accordance with the invention.

Ideally, it is furthermore intended that the, at least one, substance or complex of substances be incorporated in a cream, an ointment, a milk, a gel, a suspension, emulsion and/or dispersion.

In an equally preferable version of the present invention, it is intended that at least one substance be made available through a process selected from the following group: freeze-drying, lyophilization, spray drying, roller drying, and/or vacuum evaporation.

These types of preparation result in products which dissolve without residue on contact with fluid. Products of this sort are also known as “instant” products.

It is also intended preferably that the composition or respectively, its components, be available in sterilized form.

This can, for example, result from treating the composition with ethylene oxide and/or gamma radiation. Furthermore, a separate sterilization of the dressing (e.g. with ethylene oxide) and the composition (e.g. through autoclaving) and later combining of the components under sterile conditions is also possible.

Particularly preferable in accordance with the invention, is that with a disinfecting functioning complex of substances and a composition of at least one vitamin or vitamin derivative, a metal ion, as well as a detergent, is used. This composition is a preferred example for the uppermost defined “disinfecting complex of substances.”

A composition of this type is already described in the WO2006116983 in terms of the substances. However, this only describes the use of this composition as a decontamination solution for surfaces, but not its use for disinfecting wounds. As the expert knows, agents suited to disinfection of the surface are not necessarily suited to disinfection of wounds. Examples of this are, e.g. 70% ethanol, phenol based disinfection agents, or radiation, or disinfection processes based on temperature effects. The present invention shows for the first time, and surprisingly, the effectiveness of these compounds for disinfecting wounds.

This is surprising particularly because, for the expert, the effectiveness of a combination of substances, comprised of a vitamin or vitamin derivative, a metal ion, and a detergent is not immediately apparent to the expert. Everybody knows that these components alone do not exhibit disinfection properties. In addition, the experts avoid “off label use” of certain substances for other purposes.

The composition preferably has a pH value ranging from 2-8.5. This combination of substances results in selective deactivation of micro-organisms. Particularly preferred is that the mixture has a pH value ranging from 3-7, ideally 4-6. Ideally, the composition contains a buffer as well comprised of carbonates and succinic acid derivatives. By using this buffer in the decontamination solution of the invention, the pH value of the solution, which is very acidic, due to the dissolved components, particularly the acidic vitamins, can be raised to a slightly acidic, or even neutral or slightly basic range, without loss of the dissolved metal ions.

The vitamins contained therein, or respectively, their salts or acidic derivatives are ideally selected from the group of water soluble vitamins with anti-oxidative characteristics, such as preferably vitamin C, riboflavin and niacin.

Particularly preferred thereby is notably the combination of vitamin C and vitamin E, or respectively, vitamin B12. A combination of this sort exhibits particularly synergistic effects with regard to disinfection.

The metal ions contained therein are preferably 2- and/or 3-valent ions of metals from the 4^{th} period and/or the secondary groups I, II, and VIII [translator's note: “Nebengruppe”: “secondary groups,” a German convention referring to the fourth period of the transition elements] of the periodic table of elements. They are used in their salt forms with organic and/or inorganic acids or bases. Particularly preferred are one or more compounds selected from the secondary groups VIII-XII, specifically iron, cobalt, nickel, copper or zinc.

The detergents are ideally anionic, non-ionic, amphoteric or cationic surfactants, or suitable mixtures with or among each other. In particular, alkyl ether sulfate, alkyl- and/or arylsulfonate, alkylsulfate, amphoteric surfactants, betaines, alkylamidoalkylamine, alkyl substituted amino acids, alkyl substituted imino acids, acylated amino acids, or surfactant combinations may be used. Particularly preferred are anionic and non-ionic surfactants

The specified substance combinations can be incorporated by themselves or together with the composition of the invention in a water soluble matrix, a flat pad, a protective dressing, a foam pad, a carboxymethyl cellulose pad, a dressing containing super-absorbing particles, a solution, a cream, an ointment, a milk, a dispersion, a suspension or a gel.

Further preferred disinfecting substances or complexes of substances to be used are the so-called BLIS (bacteriocin like inhibitory substances). These are proteins with antibiotic characteristics, which are produced by various types of bacteria. They are useful in the framework of a “bacterial replacement therapy,” particularly in fighting certain streptococci.

Examples of BLIS of this type are colicin from coli bacteria, nisin from Lactococcus lactis or the BLIS from Streptococcus salivarius K12. These may be obtained in dried form (similar to the dried yogurt culture available in stores), such that upon contact with moisture, particularly exudate, they become active

In this regard, it may also be intended that recombinant micro-organisms be used in the composition, which, in the course of recombination, lose the characteristic of producing and releasing BLIS or similar inhibitors to pathogenic micro-organisms.

Both the BLIS as well as, as the case may be, dried micro-organisms may, by themselves, or in combination with the composition of the invention, be incorporated in a water soluble matrix, a flat pad, a protective dressing, a foam pad, a carboxymethyl cellulose pad, a dressing containing super-absorbing particles, a solution, a cream, an ointment, a milk, a dispersion, a suspension, or a gel.

Furthermore, octenidine, or respectively, octenidine dihydrochloride may be used as a disinfecting substance or complex of substances. Octenidine or octenidine dihydrochloride is bactericidal with both gram-positive as well as gram-negative germs, virucidal with lipophilic viruses such as the herpes simplex and hepatitis B viruses. Furthermore, it is fungicidal, although not with spores. It is colorless and odorless, may be applied painlessly, has a wide range of effects, and a retentive effect, and is therefore particularly suitable in combination with the composition of the invention.

Additionally, peroxides, such as hydrogen peroxide as well as hydrogen peroxide substances in combination with the composition of the invention are suitable as disinfecting substances or complexes of substances.

Furthermore, other peroxides may be used as a disinfecting substance or complex of substances. Specifically, monoperphthalic acid, particularly the substance magnesium monoperphthalate (MMPP), is meant here.

In addition, polyhexanide may be used as a disinfecting substance or complex of substances. Polyhexanide is a cationic diguanidine which is very microbiocidal, but not, however, virucidal or sporocidal. Due to its good tissue compatibility, polyhexanide is particularly suited for sensitive and poorly healing chronic wounds, as well as for long-term use in cleansings as well as with semi-occlusive or occlusive applications, e.g. in keeping wounds moist, and is therefore particularly suitable in combination with the composition of the invention.

Furthermore, zinc oxide may be used as a disinfecting substance or complex of substances. Zinc oxide is antiseptic, and is also suitable as a disinfecting substance.

Further disinfecting substances or complexes of substances that may be considered are listed in the main group 33.B.1 of the red list, and are all to be considered in the contents of the disclosure of the present invention. In particular, this refers to ethanol (60-90%), 1-propanol (60-70%) and isopropanol (70-80%), boric acid, chlorhexidine gluconate, iodine tincture, Lugol's iodine, povidone iodine/PVP-I, mercurochrome, 2-phenoxyethanol, phenol (carbolic acid), thymol, hexachlorophene, triclosan, Dibromol, and/or sodium hypochlorite.

Other disinfecting substances or complexes of substances to be considered are, particularly, silver, silver colloids, materials containing silver, or respectively, materials with the ability to release silver ions, peroxide, mixtures and/or alloys of silver and copper in an organic medium which, as the case may be, are enhanced with, at least one, catalyst(s) selected from the group containing platinum, platinum dioxide, titanium, titanium oxide, and titanium dioxide, antibacterial components of honey, such as propolis, honey enzyme, royal jelly etc., octenidine, antibiotics, antimycotics such as nystatin, griseofulvin, imidazole and its derivatives such as clotrimazole or tolnaftate, antivirals and similar items.

The mixtures and/or alloys of silver and copper in an organic medium to be considered, which, as the case may be, are enhanced with at least one catalyst selected from the group containing platinum, platinum dioxide, titanium, titanium oxide and titanium dioxide, are described in reference to the disinfection treatment of oral mucosa. The oxides in question can catalyze the effects of the composition, as they produce nascent oxygen, which forms a silver oxide radical with silver, which in regard to disinfection is extremely effective.

In the sense of the claim, a composition containing at least one disinfecting substance or complex of substances may be understood to be a natural sponge. A natural sponge of this type, of the demospongiae class, exhibits growth inhibiting characteristics in regard to micro-organisms in order to provide protection from the colonization of sessile organisms. These characteristics may also be useful regarding the treatment of wounds, as a means of preventing bacterial growth in the dressing and/or wound. In addition, these sponges also exhibit growth inhibiting characteristics regarding fungi and unicellular organisms, as sponges of this type are capable of absorbing liquids, and are therefore ideal for the absorption of exudate.

Said natural sponge may be placed in thin slices, created through thermal slicing for example, on the wound. It can also be incorporated in an existing dressing, as is familiar to the applicant of the present invention from, for example, the patent applications EP1411874, EP1507498 or DE202006016821U. Furthermore, it may be intended that the natural sponge in question have super-absorbent polymers. This is particularly advantageous because the containment capacity for wound exudate may be further increased and simultaneously the growth inhibiting characteristics of the sponge prevent bacterial growth in the exudate which is absorbed.

In addition, the composition containing at least one disinfecting, or respectively, decontaminating substance or complex of substances, may also have the previously described super-absorbent polymers. If these are impregnated with a solution containing bacteria, such as a pathological substrate, they then serve to restrain, particularly, the bacteria, and prevent in this manner a recontamination of the wound by the dressing. In this case, electrostatic exchanges between the polymers and the bacteria cell walls play a role.

Furthermore, the composition, which contains at least one disinfecting, or respectively, decontaminating substance or complex of substances, may have one or more exudation promoting substance. These substances are to be understood as substances that promote exudation in a wound. The promotion of exudation is specified particularly for contaminated and or infected wounds, whereby an increase in exudation results in an improvement of the rinsing of germs and other contaminants from the wound, and as a result, in decontamination. Particularly regarding contaminations of a chemical, biological or nuclear manner, this is very beneficial.

For this, for example, petroleum based blistering compounds, such as shale oil sulfonate, described here, may be used, as well as the hygroscopics also described.

Particularly preferred here are also combinations of one or more hygroscopic and one or more detergents. This combination may be, for example, comprised of glycerin, as a hygroscopic and a non-ionic detergent such as Pluronic F68. The components may be incorporated in a dressing, which, when applied to the wound releases the components in question, thereby promoting wound exudation and supporting decontamination.

In addition, a mixture containing magnetic particles (“beads”) may also be understood as the composition containing at least one disinfecting, or respectively, decontaminating substance or complex of substances, which has a functional surface. The functional surface may contain, for example, antibodies that block surface antigens of certain pathogenic germs. Furthermore, the surface may contain agents which bind contaminants, such as bacterial toxins, cell debris and similar items, either specifically or nonspecifically.

These beads may be made available, for example, in a solution, and thereby introduced to the wound (“passive introduction”). The introduction into the wound may also be forced using a repelling magnetic field (“active introduction”) which is achieved either with a permanent magnet or an electromagnet, or by using an attracting magnetic field as well, which is applied to the back side of the respective body part to be treated.

The functional surface may consist of a silicate surface, on which proteins, such as antibodies or the like, are covalently attached. Said beads are preferably between 200 nm-1,000 μm in size, and made of ferromagnetic material, such as iron, nickel, and cobalt and their alloys.

The beads are, as described, introduced to the wound either passively or actively, where they come in contact with the exudate, remain for a defined period of time, and bond to the desired targets. Subsequently they are concentrated using either a permanent magnet or an electromagnet, and removed from the wound. For this, a bandage may be applied to the wound which has an absorbing material on the side facing the wound and a magnet on the reverse side. The beads are attracted to the magnet with their targets intact, and drawn into the absorbing material, where they remain and may be removed in conjunction with the bandage.

In the same manner, a foam dressing, an alginate dressing, a film or fleece type protective dressing, a CMC pad, and activated carbon pad, a hydro-fiber pad, a hydrocolloid bandage, a gelatin pad or cotton wadding may be applied to the wound, and the patient may be subsequently placed in the active field of a permanent magnet or an electromagnet. In this manner, said product functions as an absorbing agent for the magnetic particles and the attached targets. For this, it may be intended that said product also contain metal ions. Further designs of this inventive principle are possible, which the expert, based on these descriptions, has received sufficient technical information to be able to determine, without the need of taking any inventive steps.

Regarding certain disinfecting substances or complexes of substances, the use of the composition of the invention with a dressing that is designed such that the dressing remains and nothing migrates into the wound may also be preferred. This can be particularly useful in preventing germ growth in the, as the case may be, sterilized dressing, which would otherwise be promoted through, among other factors, the intended nutritional components such as glucose and amino acids.

In this case, the previously described silver donors are intended, although quaternary ammonium compounds (QAV) such as benzalkonium chloride, cetyl trimethylammonium bromide, cetyl pyridinium chloride or benzethonium chloride, or cationic surfactants such as distearyl dimethyl ammonium chloride or esterquats also work.

These are organic ammonium compounds, whereby all four valences of a nitrogen atom are organically bonded. QAV accumulate in cell membranes of living organisms and are able to thereby affect the function of the cell membrane. As a result of this effect, the cationic surfactants in particular can also be used as disinfecting agents. Because they exhibit, among other things, skin irritant characteristics, it is therefore intended that they remain in the dressing, and are used there particularly for the inhibition of bacterial growth in the dressing.

The immobilization preferably results from covalent bonding of the QAV to the material of the dressing.

In another, equally preferred design of the invention, it is intended that there be a composition containing at least the components of an enteral and/or parenteral dietetic composition. For this, the components contained therein are an example of the above defined “nutritional substances.”

A parenteral dietetic composition contains as a rule electrolytes, i.e. minerals, carbohydrates (usually in the form of glucose), amino acids, fat and fatty substances (lipids) as well as vitamins and trace elements, and is suited to long-term exclusive nourishment of a patient. Usually these components are found in molecular forms having lower molecular weights, which, in the case of application to a wound, make their absorption in the tissue of the wound particularly easy. In particular, proteins are not included, as they can cause complications, such as allergic reactions with parenteral infusion as well as with the application in wounds.

An enteral dietetic composition is usually more complicated than a parenteral dietetic composition, i.e. it contains components having higher molecular weights, in particular proteins.

The composition can thereby be comprised of a fully balanced dietetic composition, or a dietetic composition, as well, in which certain nutrient groups, in particular, for example, carbohydrates or roughage are removed while retaining the rest of the substances in an unaltered state, or a dietetic composition which is enhanced with other substances. In regard to this, see example 1-3.

In this manner, in order to inhibit bacterial growth, for example, glucose and other carbohydrate sources may be omitted. For the same purpose, anti-bacterial substances or complexes of substances may be included, as is explained further below.

Furthermore, the composition may contain in part the so-called Brottrunk or a Brottrunk dehydrated extract. This is a drink derived from a lactic acid fermentation of whole grain sourdough bread, and is rich in nutrients or nutrient compounds, particularly zinc, iron or magnesium as well as the vitamins A, B1, B2, B6, B12, C, D, E, biotin, niacin, folic acid, and pantothenic acid. The lactic acid bacteria used in the production of said belongs to the type Lactobacillus reuteri and is still active, due to the absence of pasteurization. Because of its low pH value as well as the living lactic acid bacteria, this substrate is able to inhibit matrix proteases (see below). Said dehydrated extract can be produced through freeze-drying, for example.

The specified dietetic composition may be used as such, or in combination with the composition of the invention in a water soluble matrix, a flat pad, a protective dressing, a foam pad, a carboxymethyl cellulose pad, a dressing containing super-absorbent particles, a solution, a cream, an ointment, a milk, a dispersion, a suspension or a gel.

In addition, it may be intended that the nutrient substance or complex of substances contains insulin, recombinant insulin, proinsulin, an insulin-like growth factor (IGF), an insulin mimetic and/or a diabetic specific, non-glucose, or -sucrose based energy source

These substances also ensure a topical nutritional treatment of a wound, and are therefore included in the above definition. In this manner, insulin applied topically results in an increased absorbance of glucose through the cells to the affected area. The same applies to proinsulin, insulin-like growth factors, or insulin mimetics, in other words, molecules that have insulin-like effects in organisms. This type of topical treatment can be shown particularly with diabetics. In this case, the cellular, insulin imparted ingestion of glucose is significantly impaired as a result of a, at least temporary, deficiency of insulin. In this regard, it is particularly significant that diabetics frequently suffer from chronic edematous wounds such as venous ulcers and the like. A topical insulin therapy can significantly promote the healing process.

In this combination, diabetic specific, non-glucose or -sucrose based energy sources can also be beneficial. This includes in the following, particularly, fructose, galactose and other non-glucose or -sucrose based energy sources (fatty acids etc.). The expert can readily find in the relevant literature other diabetic specific non-glucose or -sucrose based energy sources without the need of taking any inventive steps.

Furthermore, it is preferably intended that proteases inhibiting substances or complexes of substances are proteases inhibitors, super-absorbent polymers, and chelators for divalent cations, coated magnetic particles, collagens, and/or an acidifying substance and/or complex of substances.

Thereby, it is preferably intended that for the acidifying substances and/or complexes of substances, an active element is selected from the group containing: acids, buffers, non-pathogenic acid producing micro-organisms, probiotics and/or symbiotics.

Chronic wounds frequently have a pH value in the alkaline range, i.e. a pH value which is higher than the normal pH value of the skin (pH 5.5). The said acidifying active elements are ideally organic acids. These are preferably formic acid, acetic acid, fruit based acids such as citric acid, malic acid and tartaric acid, lactic acid, gluconic acid, α-hydroxycaprilic acid, fumaric acid, or succinic acid. The expert can readily find in the relevant literature, without resorting to any inventive steps, other suitable acids as well. A portion of these acids may be made available in crystal form. Another portion of these acids is available in liquid form.

The buffer is ideally a buffer that is effective in acids. This may be, for example, an acetic acid/acetate buffer or an aluminum-iron-buffer. Particularly preferred buffer systems are the buffer systems in the blood of mammals, specifically, the carbonic acid-hydrogen carbonate buffer system (H₂CO₃+H₂O [symbol] H₃O⁺+HCO₃⁻), phosphate buffer (H₂PO₄⁻+H₂O [symbol] H₃O⁺+HPO₄²⁻), protein buffer (buffer effect through amphoteric (plasma) proteins or, as the case may be, hemoglobin (Hb.H⁺+H₂O [symbol] H₃O⁺+Hb).

The expert can find in the relevant literature, without the need of an inventive step, other suitable buffers as well.

The said acids and buffers in combination may be incorporated as such, or in combination with the composition of the invention, in a water soluble matrix, a flat pad, a protective dressing, a foam pad, a carboxymethyl cellulose pad, a dressing containing super-absorbing particles, a solution, a cream, an ointment, a milk, a dispersion, a suspension, or a gel.

The non-pathogenic, acid producing bacteria are ideally lactic acid bacteria. For this, ideally bifidobacteria, lactococci, lactobacilli, preferably of the types Lactobacillus rhamnosus, Lactobacillus acidophilus, Bifidobacterium infantis, Bifidobacterium Longum, Bifidobacterium breve, Bifidobacterium lactis, Lactococcus lactis, Streptococcus themophilus, Lactobacillus johnsonii, Lactobacillus delbrueckii, Lactobacillus neuter, Bifidobacterium animalis, Lactobacillus plantarum, Lactobacillus casei, Lactobacillus salivarius, Lactobacillus bulgaricus, Lactobacillus acidophilus DDS-1, Lactobacillus paracasei, and/or Lactobacillus sporogenes are intended.

With all of these bacteria, it is intended that they metabolize sugar and other substrate (particularly lactose and pre-biotics, see below) anaerobically, thereby producing lactic acid (lactate). They can generate thereby a pH value between 4.5-4.0 in the surrounding environment and in this manner contribute to the inhibition of the said proteases. In addition, because they are not pathogenic (in particular, they are not inflammatory), lactic acid bacteria can competitively contribute to the inhibiting of pathogenic micro-organisms in the wound, e.g. as nutrient competitors, through the precipitation of inhibitors or by producing an acidic environment which is not suited to the specific pathogenic micro-organisms. The expert can find other suitable non-pathogenic, acid producing bacteria in the relevant literature without the need taking any inventive steps.

In this regard, it may also be intended that recombinant micro-organisms be incorporated in the composition, which, in the course of recombining lose the characteristic of forming and releasing pathogenic micro-organism inhibitors.

These non-pathogenic, acid producing bacteria can, for example, be made available in dehydrated form (similar to dried yogurt culture available in stores), such that they may become active on contact with moisture, in particular, with exudate. The said lactic acid bacteria are frequently referred to as probiotics.

The said prebiotics are substances which represent a selective nutritional basis for the said non-pathogenic, acid producing bacteria, and thereby promote their reproduction, or, as the case may be, promote existing pathogenic germs. They are in, for example, chicory, black salsify, Jerusalem artichoke, and many other unprocessed, or minimally processed vegetable foodstuffs which contain prebiotics. Examples of prebiotics are fructooligosaccharide, insulin, transgalactooligosaccharide, xylooligosaccharide, mannooligosaccharide, Jerusalem artichoke juice powder or lactulose. The expert can find other suitable prebiotics as well in the relevant literature, without the need of taking an inventive step. These probiotics can exhibit an acidifying effect in and of themselves, particularly when already existing non-pathogenic, acid producing bacteria in the wound are encouraged to grow through their presence, thereby generating an acidic environment.

Symbiotics is a general term for the mixture of probiotics and prebiotics.

The micro-organisms, which, as the case may be, are available in dehydrated form, as well as the prebiotics and symbiotics may, in and of themselves, or in combination with the composition of the invention be incorporated in a water soluble matrix, a flat pad, a protective dressing, a foam pad, a carboxymethyl cellulose pad, a dressing containing super-absorbent particles, a solution, a cream, an ointment, a milk, a dispersion, a suspension or a gel.

Said proteases inhibitors inhibit proteases in the affected area which may become harmful in the course of the healing process, particularly in pathological exudate (see above). These are, particularly, matrix metalloproteinases (MMP), whose catalytic properties are partially dependant on a respective metal ion (e.g. Ca²⁺, Zn²⁺). In particular, the metal matrix proteases MMP-1-MMP-10 are familiar. For this, ideally, the use of so-called TIMPs (“tissue inhibitors of matrix metalloproteinases”) is intended. These are proteins which contain the activity of the MMPs through specific bonds to their catalytic centers. In the regeneration phase of the wound, harmful destruction and reconstruction of the tissue by MMPs can thereby be prevented. The primary candidates for this are, in particular, TIMP-1, TIMP-2, TIMP-3, and TIMP-4.

Said chelators can bond bivalent metal ions, thereby reducing the average concentration of said cations, and as a result, reduce the activity of the metalloproteinases. These are, for example, EGTA or EDTA.

Furthermore, aside from water, super-absorbent polymers can also bond with proteins and bivalent cations. The bonding of proteins also includes, as experiments have shown, the bonding of matrix proteases. These are extracted from the exudate and bonded, thereby removing them from the affected area. The bonding of bivalent cations also contributes to the deactivation of the matrix proteases. For these reasons, super-absorbent polymers may also be regarded as a composition containing at least one proteases inhibiting substance and/or complex of substances.

The characteristics mentioned are basically known. It has also been reported that dressings containing super-absorbent polymers moistened with Ringer's solution can deactivate metalloproteinases through direct bonding or bonding with bivalent cations (Smola et al., “Polyacrylate-superabsorber inhibits excessive metalloprotease activity in wound fluid from non-healing wounds”, abstracts of the ETRS annual meeting, Pisa, 2007).

With dressings containing super-absorbent polymers moistened with Ringer's solution, cleansing agents are applied to the wound and in a cyclical process release Ringer's solution to the wound and then absorb said, thereby cleansing the wound. During the cleansing process, matrix proteases and bivalent cations are also washed out of the wound and transported to the dressing, where they are bonded to the super-absorbent polymers to be found there. Due to the passive cleansing principle, this is however a very lengthy process, which requires more time than the period of time in which the dressing is applied to the wound.

In contrast, the use of super-absorbent polymers in a basically dry, absorbent dressing, as is suggested here, for the first time, by the inventor, results in a net absorption of fluid, particularly exudate, and thereby a much faster bonding of matrix proteases and bivalent cations.

Furthermore, a mixture containing magnetic particles (“beads”) may also be understood to be a composition containing at least one proteases inhibiting substance and/or complex of substances, which has a functional surface. Said functional surface may contain antibodies that block one or more matrix proteases. Additionally, said surface may contain a substrate for matrix proteases, such as collagen (particularly collagen type IV), to which proteases freely bond through their substrate bonding regions.

The active principle, as well as any material designs, has already been described in the preceding. The beads are introduced to the wound either passively or actively, where they come in contact with the exudate, remaining for a period of time and bonding to the desired matrix proteases. Subsequently, using either a permanent magnet, or an electromagnet, they are concentrated, and removed from the wound. For this, a bandage may be applied, for example, to the wound, with an absorbent material facing the wound, on the back of which a magnet is attached. The beads are attracted to the magnet with their targets intact, and arrive in the absorbent material, where they remain and may be removed from the wound together with the bandage. A vacuum therapy may also be advantageous in combination with the previously mentioned use of magnetic particles.

For the first time, using the described mechanics of magnetism, specific enzymatic activity as well as mechanisms relevant to enzyme kinetic inhibition are combined in the field of wound treatment, and in combination, allow for manipulations of the healing process reaching a depth in the tissue which otherwise could only be achieved with surgery. An incision in healthy tissue, or application through systemic means, seems comparatively unfavorable, even when it is not known how deeply the influence reaches.

The construction of the combination over a clasp shaped, hobble-type ring construction with opposing, identical or temporarily subsequently arranged polarization is conceivable.

The aim is to use magnetic forces in existing interstitial pathways in order to transport substances to the regions below the surface of the wound which have the desired effect on the healing process through controlled characteristics. In this manner, a directed transportation of substances into the depths of the tissues and back again is obtained. Using extra substrate in the form of collagens that bond to the proteases and can thereby be subsequently removed magnetically also forms a possibility. Hygroscopic substances, surfactants, nutrients, all classes of pharmaceuticals and groups of substances (=for the treatment by medicinal complexes suited to diseases) as well as other substances and complexes may be introduced which are permanently bonded to magnetic particles.

The substances which are introduced may be applied to the wound surface using various means; for example, through aerosols, fluids or granulates.

These combinations may be capable of releasing substances, such as medicines, to the depths of the tissues, in order that they are not only active on the surface, but in addition, pharmaceutically effective processes, imaging processes with contrasting agents, antibiotic measures, cytostatic steps, immune system modulations, acidifying the affected area or formation of complexes may also be initiated.

Furthermore, the use of collagens by themselves can also have a proteases inhibiting effect, which has to do with the principle of competitive inhibition. The basis of this principle is that proteases offer an excess of a substrate, collagen, which are attacked by said first. They thereby perform their function either not at all, or in a reduced manner within the affected area. The use of collagens, however, also has other benefits which will be explored further below.

In addition, the composition may also contain analgesic substances, i.e. pain relievers. For this, essentially all of the substances that are listed in the main group 05 of the so-called “red list” may be considered. Particularly preferable thereby are specifically anti-inflammatory substances such as, so-called COX inhibitors or NSAID (non-steroidal anti-inflammatory drugs), as well as, for example, propionic acid derivatives, such as naproxen, ibuprofen, ketoprofen, fenoprofen, flurbiprofen, dexibuprofen, or tiaprofenic acid, acetic acid derivatives such as diclofenac, alclofenac, etodolac, aceclofenac, sulindac, or indometacin, heterocyclic acetic acids such as ketorolac, arylalkanoic acids such as tolmetin, N-phenylacetic acids such as mefenamic acid or flufenamic acid, salicylates such as acetylsalicylic acid (aspirin), salicylic acid, or diffunisal, pyrazolone derivatives such as phenylbutazone, oxicam derivatives such as piroxicam, tenoxicam, meloxicam, or lornoxicam, enolic acid derivatives such as aminopyrine or antipyrine, phenols such as acetaminophen and similar items. In addition, there are the COX-2-inhibitors such as rofecoxib, lumiracoxib or celecoxib.

Furthermore, substances which are not anti-inflammatory may also be used as pain relievers, such as, for example, opiates, local anesthetics such as lidocaine, mepivacaine, prilocaine, procaine, syntocaine, tetracaine, Gingicaine, articaine, bupivacaine, butanilicaine, chloroprocaine, or for example, polidocanol.

In addition, the composition may contain anti-inflammatory substances that, as the case may be, exhibit secondary analgesic properties, such as, for example,—aside from the above named, which in part are also anti-inflammatory analgesics—hormones, particularly cortisone and corticoids, specifically glucocorticoids (e.g. cortisone, cloprednol, prednisone, prednisolone, methylprednisolone, deflazacort, fluocortolone, triamcinolone, dexamethasone, betamethasone) and mineralocorticoids (e.g. aldosterone, desoxycorticosterone, fludrocortisone).

As a rule, it may be beneficial to overdose the wound beyond the acute treatment needs with said substances or complexes of substances—particularly the nutrients, disinfectants and/or, as the case may be, the analgesics—as, for example, portions of the substances will remain in the dressing. This overdosing serves, however, not to resolve the overall insufficiency of the nutrients in a patient, or to prevent systemic sepsis, because a systemic effect, as is mentioned above, is neither intended nor desired. Nevertheless, the selected dosage may be higher than the dosage, as will be explained in the following, which is the recommended oral or enteral daily dosage. This is useful in particular because, for example, in the end the application of the composition in a dressing should frequently remain on the dressing for a longer period of time.

If it is the case that compositions are used, for example, that contain a dietetic composition, it is particularly intended that the amount used, for example, per dressing (e.g. foam pad, or dressing containing SAP) lies in the range between 10% and 200% of the DGE (Deutsche Gesellschaft für Ernährungsmedizin[: German Society for Nutritional Medicine]) recommended daily dosage. It is particularly preferred that this lie in the range between 30% and 100% of the recommended daily dosage.

The said composition can, thereby, be incorporated in the dissolved form of dressing, for example, or, respectively, incorporated in the dressing in advance. Alternatively, the composition may be incorporated in the dressing in its dehydrated form.

In the case that, for example, compositions are used which contain disinfecting substances, it is ideally intended that the amount used, per dressing, for example, be increased by a factor of 10 over the recommended daily dosage per kg bodyweight.

In additional preferred versions, the composition contains in addition, one or more substance(s) which are selected from the group containing orthomolecular nutrients, nutraceuticals, phytochemicals, antioxidants, growth factors, petroleum based blistering compounds, methylxanthines, tannins, tacrolimus, pimecrolimus, ATP, urea, sympathomimetic drugs, parasympatholytics, activated carbon, octenidine, polyhexanide, homeopathic remedies, Q10, thickening agents, karaja, pectin, agar, aloe vera, hemostyptics, animal saliva such as maggot or canine saliva, spider web proteins, collagen, hygroscopics, glycerin, biofilm harming substances, triacetin, zinc oxide, light absorbing components, odor inhibitors, gelling agents, exudation promoting substances, swelling reducing agents, radical scavengers, and/or honey or, respectively, its components. Most of these components belong to the definitions given above of nutrients, disinfectants, and/or proteases inhibiting substances or complexes of substances.

Orthomolecular nutrients in the framework of the concept of orthomolecular medicine, an alternative medical process largely influenced by Linus Pauling, are nutrients which are used, particularly vitamins and minerals, and may be administered in part in the intended high-dosage dosage-regimen.

Nutraceuticals are nutrients which are enriched with additional substances (nutrition enriching agents), which should have a positive effect on health. These additives are primarily vitamins, minerals, bacteria cultures, and unsaturated fatty acids.

Phytochemicals are secondary plant material, in particular; these may be carotenoids, polyphenols and sterols. Among other properties attributed to said, are those of antioxidants and the fighting of free radicals, as well as the promotion of immunity and the reduction of cholesterol levels.

Antioxidants are substances which prevent the oxidation of sensitive molecules, particularly DNA and proteins. They usually function as radical scavengers. Antioxidants can be categorized as “antioxidants”, “reducing substances” and “antioxidants with synergetic effects.” A definition for the so-called true antioxidants is the mechanism whereby the chain reaction resulting from the scavenging of free radicals is blocked. Examples of such antioxidants are BHA and BHT. In contrast to this, for example, ascorbic acid functions as a reducing agent by allowing lighter oxidation than that of the molecule being protected, thereby protecting said. Sodium EDTA belongs to this last group of synergistic antioxidants, for example, in that it enhances the antioxidant effect by bonding with metal ions.

In the framework of the present invention, the following antioxidants are to be taken into consideration: Antioxidants belonging to the vitamin E group, carotenoids, particularly lycopene and β-carotene, glutathione, transferrin, albumin, ceruloplasmin, hemopexin, haptoglobin, antioxidant enzymes, particularly superoxide dismutase (SOD), glutathione peroxidase (GPX), and catalase, tin chloride, ascorbic acid (vitamin C) and its derivative sodium L-ascorbate, calcium L-ascorbate, isoascorbic acid, sodium isoascorbate, and ascorbyl palmitate, butylated hydroxyanisole, butylated hydroxytoluene, calcium disodium EDTA, gallates, particularly propyl gallate, octyl gallate, and dodecyl gallate (lauryl gallate), lecithin, lactic acid, polyphosphates such as diphosphate, triphosphate, and polyphosphate, sulfur dioxide, sodium sulfite, sodium bisulfite, potassium sulfite, calcium sulfite, calcium bisulfite, potassium bisulfite, selenium, tocopherol (vitamin E), alpha-tocopherol, gamma-tocopherol, delta-tocopherol, tin II-chloride, citric acid as well as sodium citrate, calcium citrate, and reducing agents such as acetylcysteine.

As growth factors, growth relevant polypeptides are indicated, which are transferred from one cell to another as a signal, thereby transferring information relevant to growth. They particularly play a role in the development of multi-cell organisms. The important growth factors are:

Transforming growth factor Beta (TGF-B)

Granulocyte-colony stimulating factor (G-CSF)

Granulocyte-macrophage colony stimulating factor (GM-CSF)

Nerve growth factor (NGF)

Neurotrophins

Platelet-derived growth factor (PDGF)

Erythropoietin (EPO)

Thrombopoietin (TPO)

Myostatin (GDF-8)

Growth differentiation factor-9 (GDF9)

Basic fibroblast growth factor (bFGF of FGF2)

Vascular endothelial growth factor (VEGF)

Platelet derived growth factor (PDGF)

Epidermal growth factor (EGF)

Hepatocyte growth factor (HGF)

Petroleum based blistering compounds have an exudation promoting effect as well as a necrolytic effect. For this reason, they may also function in particular as decontaminants. One understands under this term the so-called black blistering ointments in particular, such as ichthyol based compounds, but also shale oil sulfonates. Shale oil sulfonates such as ammonium bituminosulfate, for example, are water soluble through a sulfonation process. The said compounds may be incorporated in accordance with the invention in a fatty or aqueous component of the covering of the dressing, and be applied as the first contact layer to a wound.

Methylxanthines are a group of alkaloids, which are usually used as mild stimulants as well as for treating bronchial asthma. They include caffeine, theophylline, and theobromine. Xanthines are purine derivatives. They have a constricting effect and tend to reduce swelling, so that, as the case may be, edema in the affected area is reduced, and nutritional, disinfecting and/or proteases inhibiting substances are not diluted unnecessarily.

Tannins function as astringents, i.e. they serve to reduce edema, are anti-inflammatory, antibacterial, antiviral, and neutralize toxins.

Tacrolimus (also FK506 or FK-506) is a macrolide from the bacteria Streptomyces tsukubaensis. Tacrolimus is used as, among other things, a selective immuno-suppressive against rejection reactions in organ transplants. Tacrolimus is both immuno-suppressive and antimicrobial. Its effects can be compared with those of the polypeptide cyclosporine, but may be used in smaller doses. Tacrolimus intervenes in the metabolic process of T-cells, and inhibits their activity. It bonds to the cytosolic receptor, a so-called immunophilin within the target cell. The complex comprised of immunophilin and tacrolimus adheres to the serine/threonine-protein phosphatase calcineurin. Calcineurin is thereby rendered inactive. The same basically applies for the substance tacrolimus.

ATP is a nucleotide, formed from the triphosphate of the nucleoside adenosine, and as such is an energy rich component of the nucleic acids DNA and RNA. ATP is however also the universal form of directly available energy in every cell and at the same time an important regulator of energy providing processes. ATP can be released from energy stores (glycogen, creatine phosphate) as it is needed. By adding ATP to the composition of the invention, an energy source free of glucose is made available, and is particularly useful in treatments where diabetes is present for improving the energy balance of the cells.

Urea has a high capacity for bonding with water and also exhibits keratolytic properties. In addition, it serves as a source of moisture for fighting atopic eczema and lichen diseases and is therefore particularly suited for use in a composition in accordance with the invention.

Necrolytes are agents which eat away at necrotic tissues. These may, for example, be the petroleum based blistering compounds described here. Other possible necrolytic agents are, for example, urea or animal saliva, both of which will be described below.

Sympathomimetics have a stimulating effect on the sympathetic portion of the autonomic nerve system. They affect an increase in blood pressure and pulse rate, a dilation of the bronchial passage, an overall improvement in performance and an increase in energy consumption. In combination with the composition of the invention, these substances reduce swelling as well as edema.

Parasympatholytics are medicines which counteract the action of the parasympathetic nervous system. The therapeutic use of parasympatholytics is complicated by insufficient organ selectivity. In this manner atropine, as a medicine for chronic obstructive bronchitis, promotes not only dilation of the bronchial tract, but also stimulates the heartbeat, dilation of the pupils, and a contraction of the smooth muscles. Use of these substances has a comparable effect to the sympathomimetics described above.

Activated carbon is a fine-grained carbon with a large internal surface, which is used in, among other things, chemistry, medicine, water and waste treatment as well as ventilation and air conditioning technology. When incorporated in a composition in accordance with the invention, it can contribute to bonding with toxins arising from metabolic processes and germs, and thereby, cleansing of the affected area.

Q10 or coenzyme Q10 is a quinone derivative with lipophilic terpenoid side chains, structurally related to vitamin K and vitamin E. Coenzyme Q10 is an essential electron and proton vector between the complex I or complex II and the complex III of the respiratory chains and can support the energy metabolism of the cells in the affected area through resorption with nutrients in the framework of a composition in accordance with the invention.

Thickening agents are added to solutions—as a rule, aqueous solutions—in order to increase their viscosity. They are mainly able to bond with water. Through extraction of unbonded water, the viscosity is increased. After a certain point has been reached, characteristic for each type of thickening agent, additional moisturizing effects occur which usually lead to an over proportional increase in viscosity. Thickening agents in combination with the composition of the invention allow for an adaptation to the surface of the wound, and a maximization of the resorption surface.

Suitable thickening agents are, for example, karaya (Indian tragacanth, karaya gum, E 416), a natural gum comprised of carbohydrates and galacturonic acids (secretion of the Indian sterculia tree), alginic acid, agar, carrageen, locust bean gum, guar gum, tragacanth, gum Arabic, Xanthan gum, karaya, tara gum, gellan, pectin, cellulose, cellulose ether, carboxymethyl cellulose, hydroxypropyl cellulose, hydroxypropyl methylcellulose, methyl cellulose, methyl ethyl cellulose, modified starch, egg yolk, roux, sago, and starch.

Pectins are vegetable polysaccharides, or specifically polyuronides, which for the most part are comprised of α-1,4-glycosidically linked D-galacturonic acid units. Many micro-organisms are able to metabolize pectins. Due to their ability to create gels, pectins may also be used as a thickening agent in the manner indicated above. In addition, they are capable of functioning as chelating agents in the detoxification of heavy metal poisoning, and are therefore particularly suited for use in the framework of a composition in accordance with the invention.

Aloe vera is a plant from the aloe family which is produced in a gel of the same name, also called acemannan, having a long chain polysaccharide. This substance stimulates the immune system in in-vitro experiments, protects the cell membranes, and is antibacterial, antiviral, and antimycotic. This substance is absorbed into the body particularly well through the gastro-intestinal tract, and can also be used, however, in the affected area. In addition, aloe vera contains minerals (calcium, magnesium, zinc, selenium, and others), vitamins, amino acids, and secondary plant substances (flavonoids). The term “aloe vera,” for the purposes of the present invention, may also refer to aloe vera extracts, the substances of which may be more easily absorbed by cells in the affected area.

Hemostyptics or haemostatics are substances which stimulate hemostasis, thereby making the healing process possible; in particular, vitamin K, coagulating factors (e.g. factor VIII, factor IX), trans-4-aminomethyl-cyclohexan-carboxylic acid, etc.

The term “animal saliva” refers particularly to canine saliva and maggot saliva. Canine saliva, aside from having disinfecting agents, also contains necrolytic and granulation promoting substances, and in this manner, can accelerate the healing process. Maggot saliva is the saliva from insects used in the treatment of wounds, particularly maggots (notably, the common green bottle fly, Lucilia sericata). Said insects are able to cleanse chronic wounds of necrotic tissue and bacterial waste. Aside from the removal of wound debris and bacteria, the healing process and the regeneration of fresh tissue is stimulated and promoted by substances, particularly enzymes, which are contained in the saliva of the common green bottle fly.

With said saliva, it may be particularly intended that it be produced through recombinant processes, i.e. genetically.

Spider silk is comprised of long chain protein molecules. The threads produced in the salivary glands of spiders, due to the special molecular arrangement of the amino acids involved, are very flexible, extremely resilient, highly tensile, and at the same time highly elastic. The gossamer filaments are light and water tight, having, however, a high capacity for water absorption which is comparable to wool. They are resistant to microbial attacks and are therefore ideally suited for use in the composition of the invention, for example as sheathing material for a dressing containing the composition, or as a particulate, disinfecting substance.

The term may also refer to synthetically, particularly recombinantly, produced spider silk.

Collagen is a structural proteins present in humans and animals in the connective tissue. In the human body collagen is the largest portion of protein, comprising over 30% of the total mass of all protein. Collagen is suited, in combination with the composition of the invention, as a Ca²⁺ scavenger for the reduction of proteases activity, particularly in combination with vitamin C. In addition, they also inhibit proteases, as is described above, in that they metabolize said.

Hygroscopy, in chemistry and physics, is the property of a substance (a hygroscopic) of bonding moisture in its vicinity (for the most part in the form of vapor in humidity). Hygroscopics can also, in particular, absorb exudate, and are therefore particularly suited for use in combination with the composition of the invention. Examples are super-absorbent polymers, glycerin, silicates such as silica gel, melissic acid and similar items. Said hygroscopics may also have, in particular, an exudation promoting effect, and thereby promote and enhance decontamination of the wound.

Triacetin (glycerin triacetate) is an ester compound comprised of glycerin and acetic acid. It is antimicrobial, and is used as a softener, and due to its hygroscopic properties is also used as a moisturizing agent.

Biofilms are comprised of a thin layer of mucus (film) in which micro-organisms (e.g. bacteria, algae, fungus, protozoa) are embedded. Biofilms are created when micro-organisms colonize on boundary surfaces. They form for the most part in aqueous systems, either on the surface of the water, or on a boundary surface of a solid. In more than 60% of all bacterial infections, the pathogens protect themselves from the immune system through the formation of biofilms. This includes microbial contamination and colonization of catheters, implants and instruments. Substances which destroy biofilms are, for example, abrasive substances, such as lime powder, detergents or expectorants, agents with particularly reducing effects, which can break up disulfide bonds. Their use in combination with the composition of the invention enables the reduction of germ colonization and thereby an improvement in the supplying of nutrients and/or prevention of infection to the wound.

Furthermore, it may be intended that the composition of the invention also contain swelling reducing substances such as, for example, red eyebright (Euphrasia officinalis) extract, common sage (Salvia officinalis) or cowslip (Primula veris), vasoconstrictors such as oxymetazoline hydrochloride or xylometazoline hydrochloride or anti-edemas. These may be helpful in reducing swelling in the affected area and to make edematous fluid available, in order that they may be absorbed by an absorbent dressing.

Furthermore, it may be intended that the composition of the invention contain light absorbing components. These help to prevent the loss of structural integrity in light sensitive components (e.g. zinc oxide, vitamins).

Pigments, such as titanium dioxide for example, are particularly good components for light absorption. These can be incorporated in the sheathing or together with other components such as in a solution, an emulsion or similar items as well. In addition, they may be included in the packaging of products. Said may, however, be stored in the dark in general, in order to protect the contents contained therein from light.

Odor inhibiting substances absorb malodorous substances, restrain them, or prevent their existence, thereby improving the quality of life of the patients treated with the composition of the invention. These may be, for example, activated carbon, herbal extracts, perfumes and similar items.

In general, all of the disinfecting substances or complexes of substances have odor inhibiting properties.

Furthermore, the composition of the invention may contain gelling agents, such as, for example, agar, gelatins, acrylamides, agaroses, or UV-curable gelling agents. Using these, a gel may be formed on the wound, which on one hand acts as a cover for the wound, ensuring that the wound remains moist and protected, and on the other hand, acts as a carrier and donor of the substances or complexes of substances named, and ensures a supply of said to the wound.

Radical scavengers deactivate free radicals, which otherwise place biological tissue under oxidative stress, and initiate chain reactions which can generate damage to cells and tissues, particularly changes in the cellular DNA. These may be, in particular, epigallocatechin gallate, superoxide dismutase, glutathione peroxidase, vitamin A, vitamin C, vitamin E, coenzyme Q10 and anthocyanins Bilirubin and uric acid are also able to neutralize free radicals, as well as the hormone melatonin. Radical scavengers are also frequently antioxidants. Particularly ideal thereby is notably the combination of vitamin C and vitamin E. A combination of this sort exhibits a particularly synergistic effect in regard to the antioxidative effect.

Honey consists 70-80% (mass) of inverted sugar, and contains, furthermore, enzymes, vitamins, amino acids, pollen, flavorings, and minerals. It has an antibacterial effect and is also hygroscopic, and for these reasons is particularly beneficial in combination with the composition of the invention.

Furthermore, it is particularly intended that there be one or more vitamins, selected from the group containing vitamin B12, vitamin D, vitamin C vitamin B1, vitamin B2, vitamin B6, niacin and/or folic acid in the vitamins.

An insufficiency of vitamin B12 (cobalamin) can lead to pernicious anemia (Perniziosa), a disease in the blood count and funicular myelosis. The causes of these insufficiencies may be an insufficient supply of nutrients, as has been observed with vegans, or insufficient resorption. With insufficient receptivity in the gastro-intestinal tract, the organism is lacking the intrinsic factor in its gastric juices, a glycoprotein which is produced by the parietal cells of the stomach and is essential for the metabolism of vitamin B12. The intrinsic factor binds cobalamin in a complex protected from the digestive system, and in this manner enables it to be transported in the stomach cells whereby vitamin B12 is able to arrive at the external tissues by bonding with other proteins (transcobalamin). A disturbance in the absorption in the terminal ileum may lead to insufficiency. Although a direct link to the healing process is unfamiliar to some sources, vitamin B12 is however one of the vitamins that typically need to be supplemented in older people.

Vitamin D is a collective name for a group of fat soluble vitamins which have numerous physiological effects. Its main representative in humans, vitamin D3 (or cholecalciferol) is a prohormone which the body produces in the skin with the aid of UVB light or can be supplied nutritionally.

Vitamin C is an organic acid. Because it is easily oxidized, it has antioxidant properties. Its most important property is the physiological function as a vitamin. Insufficiency can result in scurvy in humans. Vitamin C is a radical scavenger and exhibits antioxidant properties (it functions, in other words as a reduction agent). It is an important co-factor in the hydroxylation reaction and, among other things, enables the body to produce its own collagen thereby. Furthermore, it plays an important role in the production of amino acids. It protects other important metabolites and the genotype from oxidation through its antioxidant effects, or, respectively attacks from free radicals, which in the end means it provides protection to the cell from damage and thereby from cancer. Together with niacin and vitamin B6, vitamin C controls the production of L-carnitine, which is needed for the burning of fat in the musculature. In addition, it improves resorption of iron in the small intestine.

Thiamin or vitamin B1 is a water soluble vitamin in the B-complex having a weak, but characteristic odor and is particularly essential to the function of the nervous system. Vitamin B1 is necessary for the burning of carbohydrates, whereby it consumes itself as a co-enzyme. As the brain and the nerve cells are dependent on energy from carbohydrates, an insufficiency of thiamin particularly affects all brain and nerve functions.

Vitamin B2 or riboflavin serves as a preliminary step for flavin co-enzymes (FAD, FMN), which play a particularly major role in oxidoreductases, for example in citric acid cycles. It assumes a central role thereby in metabolism. Riboflavin dissolves poorly in water, is sensitive to light, but is very resistant to heat. It contributes to a smooth complexion, and is involved in the regenerative mechanisms of the skin.

The phosphorylated vitamin B6 derivatives act as co-enzymes in approximately 100 enzymatic reactions. Nearly all reactions take place in amino acid metabolism. The pyridoxal phosphate (PLP or PALP) (a pyridoxine derivative) assumes another important function as a co-factor in the synthesis of Δ-aminolevulinic acid, an intermediary product in the endogenous heme synthesis. Also noted is the participation of pyridoxal phosphate as a co-factor in the breakdown of animal starch (glycogens). Insufficiency results in the existence of dermatitides and growth disorders.

Niacin or nicotinic acid is a carboxylic acid of pyridine. Nicotinic acid is present in all living cells and is stored in the liver. It is an important building block of various co-enzymes (NAD, NADP) and is of central importance in the metabolism of proteins, fats, and carbohydrates. It is less sensitive to heat, light, and oxygen than other vitamins in the B family. Nicotinic acid participates in the metabolism of proteins, fats, and carbohydrates. In the co-enzyme form NAD/NADP and their reduced forms NADH/NADPH, the so-called reduction equivalents, nicotinic acid is involved, for example, in the citric acid cycle and the respiratory chain. It is an antioxidant, and is involved in numerous enzymatic processes. Nicotinic acid is important for the regeneration of skin, muscle, nerves and DNA.

Folic acid is sensitive to light, oxygen, and heat, as well as being water soluble. An insufficiency of folic acid in the body affects the blood count in that it may lead to a hyperchromatic macrocytic anemia.

Due to their metabolic-physiological characteristics, the vitamins named here, in particular—either alone or in combinations—have a significant influence on the healing process, specifically because they improve the local nutritional situation, and thereby contribute to an improvement of the local cell metabolism.

Particularly preferred thereby is notably the combination of vitamin C and vitamin E. A combination of this sort has synergistic effects in particular.

The substances named can be, without exception, in combination with the composition of the invention incorporated in a water soluble matrix, a flat pad, a protective dressing, a foam pad, a carboxymethyl cellulose pad, a dressing containing super-absorbent particles, a solution, a cream, an ointment, a milk, a dispersion, a suspension, or a gel.

In accordance with the invention, in addition a dressing is intended which contains a foam pad and/or super-absorbent particles, characterized in that this contains a composition in accordance with the preceding description.

In addition, a dressing is intended containing super-absorbent particles, characterized in that this contains a composition in accordance with the preceding description.

Furthermore, a kit is intended of various compositions in accordance with the preceding description, various pads in accordance with the preceding description or various dressings in accordance with the preceding description, which is characterized in that the various compositions, pads or dressings of the kit are in each case appropriate for various phases of the wound.

In observing the healing processes, four phases of the healing processes may be distinguished, specifically: the inflammatory or exudative phase (cleansing phase), the granulation phase, the epithelization phase, and the reparative phase. The epithelization phase and the reparative phase are occasionally considered as one.

1. Cleansing Phase

Particularly with acute wounds, hemostasis is of primary importance directly after the wound has occurred. The complement system (coagulation cascade) is activated, and loss of blood is limited by thrombocytes and aggregation of fibrin. This is supported by a simultaneous vasoconstriction. Subsequently, there is an increased release of histamines and serotonins by the damaged cells. The resulting recurrence of vasodilation with a simultaneous increase in vascular permeability leads to the formation of wound edema. This can be seen by the reddening and heating of the skin as well the resulting pain the patient experiences. In a later part of the phase, the cleansing of the wound is of primary importance. Edematous fluid is exuded from the affected area in the form of protein rich exudate, cleaning the wound. At the same time, leukocyte infiltration in the affected area is stimulated. This last aspect results as well due to mechanical cleansing of germs and excess tissue, supported by biochemical processes for defense against germs and the active removal of irreversibly damaged tissues. The exudative phase serves to prepare the affected area for the following phases of the healing process. This particularly applies to the processes for chronic wounds, which do not always exhibit the bleeding mentioned at the beginning.

2. Granulation Phase

If the wound exhibits the corresponding prepared areas resulting from the exudative phase, then within the subsequent two to four days fibroblasts begin to collect and stroma is formed, also known as granulation tissue. The regeneration of tissue develops along the fibrin matrix of the blood coagulation and uses the supply of nutrients resulting from the simultaneous regeneration of blood vessels (angiogenesis).

3. Epithelization Phase

Through the initiation of contraction of the edges of the wound, the amount of necessary regeneration of tissue is reduced and epithelization phase begins. At this point, a regeneration of tissue begins at the edges of the wound, whereby keratinocytes or basal cells may be involved.

4. Reparative Phase

The epithelization is completed in the reparative phase, provided a corresponding prepared wound surface exists. The granulation tissue is converted to scar tissue. At this point the vessels recede, and over the course of months or even years, a hard, fibrous scar tissue forms. In this phase, proteases (particularly matrix metalloproteinases) may have a particularly harmful effect.

The wound has different requirements during each phase, which are met by the kit of the invention. In this manner, a kit may be assembled from dressings equipped with four different compositions. A kit of this sort is described in the following table 1:

TABLE 1

| | Requirements for treatment | Ideal substances anticipated in the |
|---|---|---|
| Phase | and/or care | composition of the invention |
| 1 | 1. Absorption of exudate | 1. Super-absorbent particles |
| | 2. Inhibition of bacteria | 2. Immobilized silver donors in the |
| | growth in the dressing | dressing |
| | 3. Disinfection of the wound | 3. Mixture of vitamins and/or vitamin |
| | 4. Inhibition of proteases | derivatives, metal ions, surfactants |
| | | 4. Lactic acid bacteria and probiotics |
| | | and/or TIMP and/or acids |
| 2 | 1. Nourishing of the wound | 1. Nutritional substances |
| | 2. Disinfection of the wound | 2. Mixture of vitamins and/or vitamin |
| | | derivatives, metal ions, surfactants |
| 3 | 1. Nourishing of the wound | 1. Nutritional substances |
| | 2. Disinfection of the wound | 2. Mixture of vitamins and/or vitamin |
| | | derivatives, metal ions, surfactants |
| 4 | 1. Disinfection of the wound | 1. Mixture of vitamins and/or vitamin |
| | 2. Conditioning of the | derivatives, metal ions, surfactants |
| | wound surface | 2. Urea, aloe vera |

In addition, in accordance with the invention, it is intended that such a kit be used for external, non-systemic, topical care and/or treatment of wounds to human or animal bodies, whereby various components of the kit are used during various phases of the wound.

In deviating from this approach, it may naturally also be intended that a composition, particularly a pad, a protective dressing and/or a dressing is provided which is configured such that the substances or complexes of substances contained therein are appropriate to the wound in each phase of the healing process. The background for this approach is that there are wounds which are simultaneously in different phases of the healing process at different points on the wound. It is naturally advantageous to be able to meet the needs of all of these points with only one product.

In addition, the solubility characteristics of individual substances or complexes of substances may be adjusted such that they dissolve at different points in time (i.e. in different phases of the healing process) and are released into the wound. For this, for example, different sizes of the particles may be determined, i.e. a larger particle sizes may be used for substances or complexes of substances that are to be released at a later period, and smaller particle sizes may be used for substances or complexes of substances that are to be released at an earlier period (sustained-release effect).

Alternatively, the respective substances or complexes of substances may be arrayed in degradable capsules having different degradable constants, thus releasing their contents to the wound in the appropriate phases. In this manner, the sustained-release effect can also be obtained.

In summary, it can be said that new standards in the treatment of wounds can be established through these means of wound phase appropriate care.

### ILLUSTRATIONS AND EXAMPLES

The present invention will be explained in greater detail through the presentation and discussion of the following illustrations. It should be noted thereby that the illustrations and examples have only the described character, and in no manner are intended to limit the invention.

### Example 1

### Composition of a Nutrient Composition without Proteins, as Listed in Table 2

TABLE 2

| Component | Quantity | |
|---|---|---|
| Isoleucine | 2.5 | g |
| Leucine | 3.7 | g |
| Lysine-HCL | 4.125 | g |
| Methionine | 2.15 | g |
| Phenylalanine | 2.55 | g |
| Threonine | 2.2 | g |
| Tryptophan | 1 | g |
| Valine | 3.1 | g |
| Arginine | 6 | g |
| Histidine | 1.5 | g |
| Glycine | 7 | g |
| Alanine | 7.5 | g |
| Proline | 7.5 | g |
| Malic acid | 3.065 | g |
| Glucose 1H₂O | 220 | g |
| Sodium chloride | 1.169 | g |
| Potassium chloride | 2.238 | g |
| Calcium chloride 2H₂O | 0.368 | g |
| Magnesium chloride 6H₂O | 0.509 | g |
| Zinc chloride | 0.0055 | g |
| Glycerol-1(2)-dihydrogen phosphate— | 4.592 | g |
| mixed with disodium salts | | |
| Total | 282.78 | g |

The composition meets the needs of a fully balanced parenteral dietetic composition. It contains, in the amounts listed, approximately 60% of the daily requirements of a man weighing 80 kg (176 lbs) based on the recommended daily dosage of the DGE (Deutsche Gesellschaft für Ernährungsmedizin [: German Society for Nutritional Medicine]). In the use with a dressing for wounds, this corresponds to a topical overdose. This overdosing, however, does not serve the purpose of correcting a general insufficiency in the nutritional situation of a patient or prevention of a systemic sepsis, as a systemic effect is neither intended nor desired, as is mentioned above.

### Example 2

### Composition of a Nutrient Composition in Accordance with the Invention, without Proteins and without Carbohydrate Sources

Isoleucine: 2.5 g, Leucine: 3.7 g, Lysine-HCL: 4.125 g (containing 3.3 g L-Lysine), Methionine: 2.15 g, Phenylalanine: 2.55 g, Threonine: 2.2 g, Tryptophan: 1 g, Valine: 3.1 g, Arginine: 6 g, Histidine: 1.5 g, Glycine: 7 g, Alanine: 7.5 g, Proline: 7.5 g, Malic acid: 3.065 g, Sodium chloride: 1.169 g, Potassium chloride: 2.238 g, Calcium chloride 2H₂O: 0.368 g, Magnesium chloride 6H₂O: 0.509 g, Zinc chloride: 0.0055 g, Glycerol-1(2)-dihydrogen phosphate—mixed with disodium salts 5H₂O (40/60 G/G): 4.592 g, (in mmol/l: Na⁺ 50, K⁺ 30, Ca²⁺ 2.5, Mg²⁺ 2.5, Zn²⁺ 0.04, Cl⁻ 100.11, Glycerophosphate 15).

The composition meets the needs of a fully balanced parenteral dietetic composition with the absence of glucose. This may be applied in the case of highly infected wounds in order to prevent a supply of carbohydrates to the infectious bacteria.

### Example 3

### Composition of an Additional Nutrient Composition in Accordance with the Invention

Glucose syrup, maltodextrin, vegetable oil, soy protein isolate, milk protein, inulin, guar gum, glucose, soy fiber, emulsifier: soy lecithin, magnesium carbonate, calcium orthophosphate, potassium chloride, choline hydrogen tartrate, calcium carbonate, vitamin mixture (vitamin C, niacin, vitamin E, pantothenate, vitamin B2, vitamin B6, vitamin B1, vitamin A, folic acid, vitamin K, biotin, vitamin D3, vitamin B12), sodium citrate, taurine, iron lactate, L-carnitine, zinc oxide, potassium fluoride, manganese sulfate, copper sulfate, potassium iodate, chromium chloride.

Said composition contains the following nutrients quantities, as listed in Table 3:

TABLE 3

| Component | Ø content per 100 g powder | |
|---|---|---|
| Energy | 1821 kJ/433 kcal | |
| Protein (13% energy) | 13.8 | g |
| Carbohydrates (56% E.) | 60 | g |
| Roughage | 6.2 | g |
| Fat (30% energy) | 14.6 | g |
| saturated fatty acids | 5.2 | g |
| simple unsaturated fatty acids | 6.4 | g |
| complex unsaturated fatty acids | 3 | g |
| Sodium | 260 | mg |
| Potassium | 435 | mg |
| Calcium | 260 | mg |
| Magnesium | 70 | mg |
| Phosphor | 176 | mg |
| Chloride | 385 | mg |
| Iron | 4.8 | mg |
| Zinc | 4.7 | mg |
| Copper | 435 | μg |
| Iodine | 45 | μg |
| Chromium | 20 | μg |
| Fluorine | 0.4 | mg |
| Manganese | 0.6 | mg |
| Molybdenum | 22 | μg |
| Selenium | 12.5 | μg |
| Vitamin A | 260 | μg |
| Vitamin D | 3.1 | μg |
| Vitamin E | 9.5 | mg |
| Vitamin K | 35 | μg |
| Vitamin B1 | 0.45 | mg |
| Vitamin B2 | 0.58 | mg |
| Vitamin B6 | 0.45 | mg |
| Vitamin B12 | 1.1 | μg |
| Vitamin C | 60 | mg |
| Niacin | 5.9 | mg |
| Folic acid | 70 | μg |
| Pantothenic acid | 2.6 | mg |
| Biotin | 16 | μg |
| Choline | 77 | mg |
| Taurine | 24 | mg |
| L-carnitine | 6.5 | mg |
| Inositol | 18 | mg |

The composition meets the needs of a fully balanced enteral dietetic composition. In the quantities listed it corresponds to approximately 30% of the daily requirements of an 80 kg (176 lbs.) man, in accordance with the recommended daily dosage of the DGE (Deutsche Gesellschaft für Ernährungsmedizin [: German Society for Nutritional Medicine]). In the use with a dressing for wounds, this corresponds to a topical overdose. This overdosing, however, does not serve the purpose of correcting a general insufficiency in the nutritional situation of a patient or prevention of a systemic sepsis, as a systemic effect is neither intended nor desired, as is mentioned above.

As the case may be, it may be intended that the carbohydrate portion (particularly glucose, but also, as the case may be, maltose, maltodextrin, isomaltose or starch) be omitted, in order to prevent a supply of carbohydrates to infectious bacteria in the case of a highly infected wound. In addition, it may be intended that roughage such as inulin or soy fiber, emulsifiers such as monoglyceride or diglyceride or soy lecithin and/or thickening agents such as guar gum be omitted.

### Example 4

### Composition of Various Disinfecting Compositions in Accordance with the Invention Containing at Least One Vitamin or Vitamin Derivative, a Metal Ion and a Detergent in 50 μg Distilled Water

TABLE 4

| Vitamin or derivative | Metal ion | Detergent |
|---|---|---|
| 100 mM vitamin C | 10 mM FeCl₃ | 0.5% SDS |
| 50 mM Vitamin E | | |
| 100 mM vitamin C | 10 mM ZnCl₂ | 2% TritonX-100 |
| 50 mM Vitamin E | | 0.2% Tween 20 |

### Example 5

### Composition in Accordance with the Invention Containing Non-Pathogens, Lactic Acid Producing Micro-Organisms as Acidifying Agents

A composition containing the following components in Table 5:

TABLE 5

| Component | Quantity |
|---|---|
| Lactobacillus acidophilus | 6.3% by mass |
| Lactococcus lactis | 2.1% by mass |
| Bifidobacterium Longum | 2.1% by mass |
| Lactobacillus rhamnosus | 1.4% by mass |
| Bifidobacterium breve | 1.4% by mass |
| Bifidobacterium bifidum | 0.7% by mass |
| Ascorbic acid | 1% by mass |
| Potato starch as carrier | Ad. 100% by mass |

The micro-organisms specified are available in freeze-dried form and are activated on contact with the wound through heat and moisture.

The composition is composed in such a manner that 2 grams of said contains at least 5×10⁸ of the respective bacteria.

### Example 6

### Composition in Accordance with the Invention Containing Non-Pathogens, Lactic Acid Producing Micro-Organisms as Acidifying Agents, Such as Prebiotics

Added to 2 g of the composition in example 5 are 5 g of a mixture of inulin (65% by mass), oligofructose (20% by mass) and Jerusalem artichoke juice powder (15% by mass).

### Example 7

### Production of a Dressing Containing SAP which Contains a Composition in Accordance with the Invention

A composition in accordance with any of the examples 1-6 is compressed together with cellulose fibers. The following quantities per 100 g cellulose fibers are used thereby, as shown in Table 6:

TABLE 6

| | | | Corresponds to |
|---|---|---|---|
| Composition of | | | portion of systemic |
| example | Quantity | | daily dosage of |
| 1 (nutritive) | 100 | g | 21% |
| 2 (nutritive) | 22 | g | 21% |
| 3 (nutritive) | 100 | g | 30% |
| 4 (disinfecting) | 50 | μg water | n/a |
| | 100 | mM vitamin C | |
| | 50 | mM vitamin E | |
| | 10 | mM FeCl₃ | |
| | 0.5% | SDS | |
| 5 (acidifying) | 2 | g | 100% |
| 6 (proteases inhibiting) | 7 | g | 100% |

| | | |
|---|---|---|
| 1 + 4 (nutritive and | See above | 21% |
| disinfecting, suited to | | |
| wound phase 3) | | |

The cellulose fibers are then replaced in a process with 50% by mass super-absorbent polymers (co-polymer of acrylic acid and sodium acrylates), as is described in the DE19750890, the contents of the disclosure of which shall be added in full to this writing, and processed in an air-laid of the dimensions 20×10 cm. Subsequently, this air-laid shall be covered on both sides with a cellulose pad, and packed in a sheath of polypropylene, having pores ranging in size between 0.1 mm and 1.0 mm. The sheath shall have an ultra-sound seal on its edges.

### Example 8

### Production of a Dressing Made of PU Foam, Containing a Composition in Accordance with the Invention

A composition in accordance with any of the examples 1-6 is dissolved in 200 ml distilled water, in the same quantities as those in example 7. Subsequently, a 20×20×0.3 cm pad of PU foam shall be impregnated with this solution, and dried.

### Example 9

### Production of a Dressing Containing a QAV

A composition in accordance with any of the examples 1-6 as well as 20 mg benzalkonium chloride is dissolved in 200 mg distilled water, in the same quantities as used in example 7. Subsequently, a 20×20×0.3 cm pad of PU foam shall be impregnated with this solution, and dried.

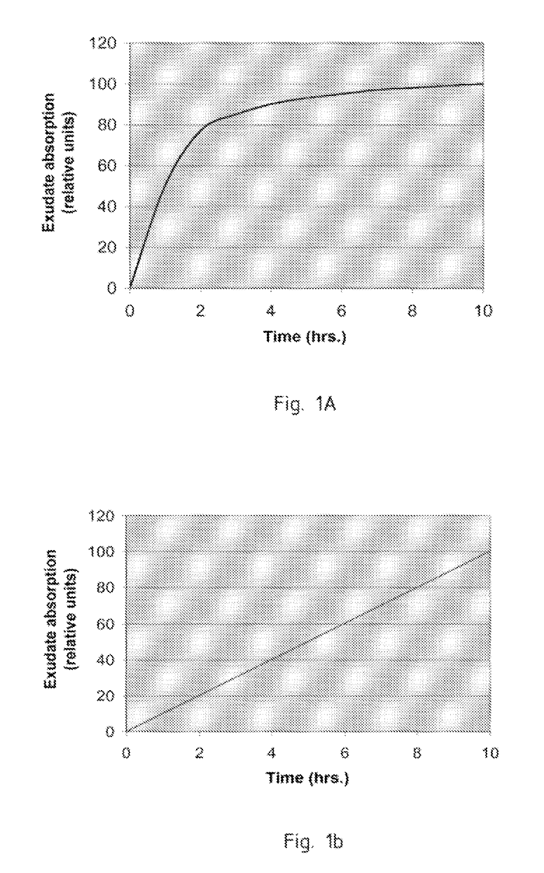

FIG. 1: Absorption of Exudation Dressing which is for the Most Part Dry, or, Respectively, a Moistened Dressing Containing Super-Absorbent Polymers.

FIG. 1A shows the absorption of exudate in a, for the most part, dry, absorbent dressing containing super-absorbent polymers, as is described in the DE10059439 as well as the WO03094813 by the applicant of the present invention. A dressing of this type has, particularly in the initial phase, an enormous capacity for absorption, which is represented by the hyperbolic curve shown therein. Due to the bonding characteristics of the super-absorbent polymers in regard to proteins, particularly matrix proteases as well as bivalent cations, these are discharged quickly from the wound, thereby promoting the healing process. This is of particular advantage when the dressings are to be changed frequently. Is shall then be ensured that at the point in time when the dressing is changed, the saturation capacity of the dressing (and thereby the maximal bonding capacity of matrix proteases and bivalent cations) has been reached.

FIG. 1B in contrast shows the exudate absorption of a dressing containing super-absorbent polymers, moistened with Ringer's solution. In this case, a cleansing body is applied to the wound, whereby in a cyclical process Ringer's solution is released into the wound and in turn absorbed from said, thereby cleansing the wound. A dressing of this sort does not exhibit a net absorption of fluid in this manner. During the cleansing process, however, the wound is successively washed and the exudate and its contents, particularly matrix proteases and bivalent cations, are absorbed by the dressing, where they are restrained by the super-absorbent polymers present therein. Due to the passive cleansing principle, this is however a very length process, which requires a longer period of time than is required for the dressing to be applied to the wound. It can therefore be the result that the dressing already is changed before it has reached its maximal saturation point.

What is claimed is:

1. A composition containing at least one disinfecting or decontaminating substance and/or complex of substances for external care and/or treatment of wounds to a human or animal body, wherein the at least one substance and/or complex of substances is incorporated in an absorbent wound dressing containing super-absorbent particles, in such way that the wound exudate absorbed by the super-absorbent particles serves as dissolving agent for the substances or complexes of substances mentioned, thus contributing to their dissolution and the introduction, or diffusion, thereof to the wound, wherein the disinfecting or decontaminating substance or complexes of substances is octenidine or octenidine dihydrochloride.

2. A composition in accordance with claim 1, characterized in that at least one substance or complex of substances is available in a water soluble form.

3. A composition in accordance with claim 1, characterized in that at least one substance or complex of substances is incorporated in a water soluble matrix.

4. A composition in accordance with claim 1, characterized in that at least one substance or complex of substances is incorporated into a flat mat.

5. A composition in accordance with claim 1, characterized in that the composition or the components of said substance are available in a sterilized form.

6. Wound dressing, containing a foam mat and/or superabsorbent particles, containing the composition according to claim 1.

7. A composition in accordance with claim 1, characterized in that the dosage form of at least one substance or complex of substances is selected such that it comprises solely a topical effect.

8. A composition in accordance with claim 1, characterized in that for at least one of the disinfecting complexes of substances, said substance consists of a composition of at least one vitamin or vitamin derivative, a metal ion and a cleansing agent.

9. A composition in accordance with claim 1, characterized in that the at least one of the disinfecting substances or complexes of substances, said consists of a bacteriocin-like inhibitory substance.

10. A composition in accordance with claim 1, characterized in that the at least one of the disinfecting substances or complexes of substances consists of coated magnetic particles.

11. A composition in accordance with claim 1, characterized in that the composition is incorporated in an absorbent dressing containing a foam pad, an air-laid, a carboxymethyl cellulose pad, and alginate pad.
